(19) United States
(12) Patent Application Publication
(10) Pub. No.: US 2004/0208905 A1
(43) Pub. Date: Oct. 21, 2004

(54) Gene delivery of a viral vector

(76) Inventors: Seppo Yla-Herttuala, Kuopio (FI); Kari Juhani Airenne, Kuopio (FI); Pauliina Lehtolainen, Kuopio (FI)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/478,978

(22) PCT Filed: May 28, 2002
(86) PCT No.: PCT/GB02/02504

(30) Foreign Application Priority Data
May 29, 2001 (WO) PCT/GB01/02383
Nov. 29, 2001 (GB) 0128620.2

(51) Int. Cl. A61K 7/00

(52) U.S. Cl. 424/401

(57) ABSTRACT

A baculovirus is used for gene therapy of a condition that can be mediated via the spinal cord or CNS.

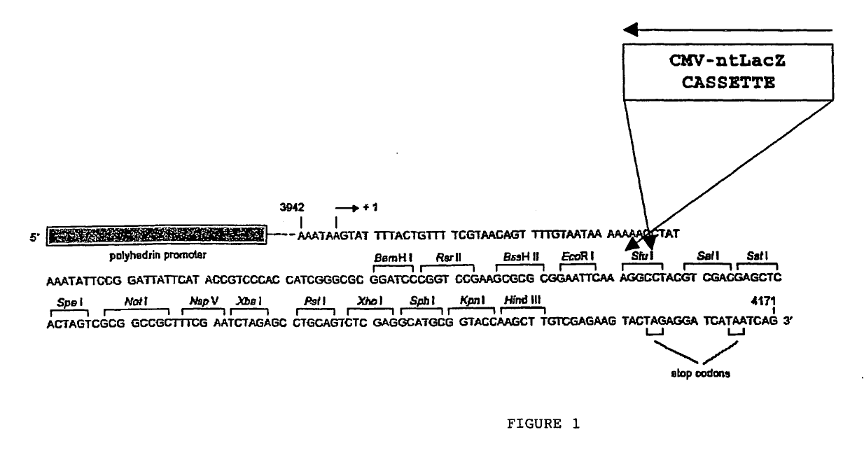

### FIELD OF THE INVENTION

This invention relates to gene delivery using a viral vector.

### BACKGROUND OF THE INVENTION

Efficient gene transfer would be a beneficial tool for the treatment of vascular diseases, such as post-angioplasty restenosis, post-bypass atherosclerosis, peripheral atherosclerotic disease, stenosis of vascular prosthesis anastomoses, and thrombus formation. Various techniques have been developed for this purpose; see, for example, Yla-Herttuala et a/, J. Clin. Invest. 95:2692-8 (1995), and Laitinen et a/, Hum. Gene. Ther. 8:1645-50 (1997).

WO-A-98/20027 discloses a periadventitial collar that can be used for arterial gene transfer during vascular surgery. However, there is a continuous need for more facile and efficient gene transfer vectors. Only a temporary expression of the transgene may be required to achieve a beneficial biological effect in cardiovascular applications; see Yla-Herttuala et a/, Lancet 355:213-222 (2000).

Baculoviruses have long been used as biopesticides and as tools for efficient recombinant protein production in insect cells. They are generally regarded as safe, due to the naturally high species specificity and because they are not known to propagate in any non-invertebrate host. Although the virions have been shown to enter certain cell lines derived from vertebrate species, no evidence of viral gene expression has been detected using natural viruses. However, the Autographa californica multiple nuclear polyhedrosis virus (AcMNPV), containing an appropriate eukaryotic promoter, is able to transfer and express target genes efficiently in several mammalian cell types; see, for example, Hofmann et a/, PNAS USA 92:10099-10103 (1995). In addition, Barsoum et at, Hum. Gene. Ther. 8:2011-8 (1997), has reported that baculovirus having the vesicular stomatitis virus C glycoprotein in its envelope significantly increases the efficiency of transduction of human hepatoma cell lines and broadens the range of mammalian cell types that can be transduced by baculoviruses. Stable transduction of mammalian cells by baculoviruses has been achieved by either including an expression cassette encoding a dominant selectable marker into baculovirus genome or by using hybrid baculovirus-adeno-associated virus vector; see Condreay eta/, PNAS USA 96:127-132 (1999), and Palombo et al, J. Virol. 72:5025-34 (1998).

Sandig et a/, Hum. Gene Ther. 7:1937-45 (1996), reported unsuccessful attempts to use baculoviruses for in vivo gene delivery in mice and rats by systemic or intraportal application as well as by direct injection into the liver parenchyma. One reason for this is presumably the inactivation of baculoviruses by the classical pathway of serum complement system.

WO-A-00/05394 discloses baculovirus vectors and their use for gene transfer to the nerve cells of vertebrates.

### SUMMARY OF THE INVENTION

It has now been found that the inactivation of baculoviruses can be avoided. In particular, it has been shown that baculoviruses are able to mediate periadventital gene transfer to rabbit carotid arteries with an efficiency comparable to adenoviruses. The ease of manipulation and rapid construction of recombinant baculoviruses, their lack of cytotoxicity in mammalian cells even at a high multiplicity of infection, their inherent incapability to replicate in mammalian cells, and their large capacity for the insertion of foreign sequences, make baculoviruses very suitable tools for in vivo gene therapy.

This invention is able to use the advantageous properties of baculoviruses, in a suitable vector, from which the gene is expressed, if administered (in or ex vivo) to a body site at which there is no blood, or which is essentially free of blood. Thus, periadventitial or, more specifically, collar-mediated local gene delivery allows gene transfer essentially in the absence of serum, thus avoiding deleterious effects of serum components. The novel method also avoids two other major problems encountered in systemic gene delivery, i.e. a rapid redistribution of the virus from the injection site and a drop in the local concentration of the virus.

In particular, it has been found that baculoviruses specifically transduced cuboid epithelium of the choroid plexus in ventricles and that the transduction efficiency was as high as 76%+14, whereas adenoviruses showed preference to corpus callosum glial cells and ventricular ependymal lining. Only a modest microglia response was seen after the baculovirus transduction, whereas the adenovirus gene transfer led to a strong microglia response. Sensitive nested RT-PCR revealed transgene expression in hindbrain and in ectopic organs including spleen, heart and lung, which indicates that some escape of both vectors occur to ectopic organs after local gene transfer to brain. Thus, baculovirus vectors can be used for local intracerebral gene therapy. The knowledge of the cell type specificity of the vectors offers a possibility to achieve targeted gene delivery to distinct brain areas. Baculoviruses seem to be especially useful for the targeting of choroid plexus cells.

Since choroid plexus cells are involved in the production of cerebrospinal fluid, they are a target for the production of secreted therapeutic proteins in the brain. It may be deduced that, by utilizing the naturally restricted cell tropism, baculoviruses provide an efficient tool for gene delivery to cerebral choroid plexus cells and may become useful for gene therapy of several types of brain disorders.

### DESCRIPTION OF THE INVENTION

Suitable delivery systems, active materials, formulations, dosages etc, are illustrated in WO-A-98/20027 and also WO-A-99/55315 (the contents of which are incorporated herein by reference). Thus, by way of example only, the delivery vehicle may be a collar or wrap. By comparison with those publications, the vector for gene delivery is a baculovirus.

Baculoviruses are of course known, and the skilled person will be able to construct any suitable vector for use in this invention. It will also be evident that the broad knowledge of baculovirus biology and AcMNPV genome will aid engineering of the improved second-generation viruses for gene transfer applications. The ease of construction, and capacity to accept large foreign DNA-fragments (>20 kbp), allows the development of baculoviruses having enlarged ortargeted cell tropism along with more stable, temporal and cell type-specific control of transgene expression. A recombinant baculovirus for use in the invention may be formulated into a medicament for therapeutic use, in known manner.

Routes and sites of administration for the invention include intra-ocular application, intra-articular application, superficial intra-dermal application, ureters, bladder, Fallopian tubes, gall bladder, spinal cord, cerebrospinal fluid compartment, pleural cavity and intraperitoneal cavity. Sites that have been used are arteries, brain and skeletal muscle, including, by way of example, mycocytes, satellite cells and regenerating myoblasts. Gene delivery may be done via direct injection or various types of catheters.

If appropriate, body parts can be made “bloodless” during surgery. This technique is often used in leg or arm surgery by putting tight pressure around arm or thigh, thus preventing blood flow. The body part may then be perfused with saline to remove blood, and baculovirus transfection can then be done.

The invention can be used for the delivery of an agonist of a VEGF receptor, e.g. described in more detail in WO-A-98/20027. Further, by suitable choice of the gene, it may be used in the treatment of cancer, e.g. in the brain.

Administration to the brain may be intraventricular or, owing to the apparent presence of specific receptors for baculovirus in choroid plexus cells, elsewhere. The gene that is delivered may be designed for enzyme replacement therapy. For example, the active agent may cause the production of NO, e.g. to treat subarachnoid hemorrhage; a suitable gene is for endothelial NO synthase. More generally, the active agent may be for any condition that affects or can be mediated via the spinal cord/CNS.

A further aspect of the invention relates to transplant organs and vessels which can be perfused with saline ex vivo and subjected to ex vivo baculovirus injection.

The following experimental work illustrates the invention.

Using essentially the same procedure as in the Example of WO-A-01/09390, this Example shows that baculovirus gene transfer works in brain and skeletal muscle. Using baculovirus/lacZ, rat brain shows positive transfection in various types of brain cells, especially in choroid plexus cells in ventricles and endothelial cells. The profile of transfected cells is clearly different from that of adenoviruses.

Further, baculovirus transfection has been demonstrated in rabbit skeletal muscle. Baculovirus encoding lacZ (1.8×10^{10.}PFU) was directly injected into the adductor muscle of NZW rabbit via a 25 G needle. The injection volume was 0.5 ml. Tissue samples were collected 7 days after the gene transfer, and X-Gal staining was performed overnight. These results clearly indicate that baculovirus can be used for transfection of several cell types in mammalians, i.e. not only arterial cells.

The accompanying drawing illustrates the construction of a nuclear-targeted β-galactosidase-encoding baculovirus transfection cassette. In principle, this is a standard public domain baculovirus with polyhedrin promoter, into which have been cloned restriction sites and the CMV-NT lacZ expression cassette. The lacZ expression cassette is oriented opposite to the polyhedrin promoter. The sequence of the CMV-nt lacZ expression cassette is in SEQ ID NO:7.

In the drawing, +1 corresponds to the transcriptional start for the polyhedrin promoter. ATT-site of original transcriptional start. The ATG was mutated to an ATT.

In Vivo Injections of Viruses

Inbred female BDIX rats (n=38) were used for the studies. Results were confirmed in Wistar rats (n=11). Rats (200-250 g) were anesthetized intraperitoneally with a solution (0.150 ml/100 g) containing fentanyl-fluanisone (Janssen-Cilag, Hypnorm®, Buckinghamshire, UK) and midazolame (Roche, Donmicum®, Espoo Finland), placed into stereotaxic apparatus (Kopf Instruments) and 20 μl of the virus in PBS/0.1% sucrose was injected during 2×10 min periods using Hamilton syringe with a 27-gauge needle. Procedure was repeated in three consecutive days. Injections of the viral vectors intracranially in the right corpus callosum were performed at the following coordinates: A) 1 mm to bregma, 2 mm to the midline, and 2.5 mm of depth (n=14 for baculovirus and n=27 for adenovirus) and B) 2 mm to bregma, 2.5 mm to the midline, and 1.7 mm of depth (n=8 for baculovirus). Rats received 108 plaque forming units (pfu) of both vectors.

Immunohistochemistry

Animals were sacrificed with CO₂ 5, 10, 14 and 21 days after the gene transfer. Rats were perfused with 1×PBS by transcardiac route for 10 min followed by fixation with 4% paraformaldehyde/0.15 M sodium-phosphate buffer(pH 7.4) for 10 min. Brain was removed and divided at the injection site into two coronal pieces. Samples from fore and hindbrain, liver, kidney, heart, spleen, lung and skeletal muscle (psoas major) were taken. Tissue samples were rinsed in 1×PBS and embedded in O.C.T. compound (Tissue-Tek, Sakura) or snap-frozen for nested PCR analysis. The LacZ activity of the sections was analysed with 5-bromo-4-chloro-3indolyi-β-D-galactopyranoside (X-Gal; MBI Fermentas) for 18 h to identify β-galactosidase positive cells. Gene transfer efficiency was calculated from 5-8 randomly selected sections at the injection site from each animal as a percent of the β-galactosidase positive nuclei of the total number of nuclei in the specific cell types (i.e. choroid plexus, ependyma, corpus callosum) from the area of 200×200 μm. Monoclonal antibodies CD31 (1:200, Dako), anti-fibrillary acidic protein (GFAP 1:400, Boehringer Mannheim) and CD11b (OX-42 1:200, Serotec) were used to identify endothelial, astrocytic and microglial cells, respectively. Avidin-biotin-HRP system and biotinylated secondary antibodieswith DAP staining were used for signal detection (Vector Elite, Vector Laboratories, Burlingam, Calif.). Sections were counterstained with Mayer's Carmalum or hematoxylin and data were collected with Image-Pro Plus software with Olympus AX70 microscope (Olympus Optical, Japan). Controls for immunostaining included incubations with class- and species-matched immunoglobulins and incubations without primary antibodies.

RT-PCR

Total RNA from spleen, liver, kidney, lung, heart, skeletal muscle and transduced brain samples was extracted using TRIZOL reagent (Gibco-BRL). Samples were subsequently treated with RQ1 RNase free DNase (Promega, Madison, Wis., USA) to eliminate DNA contamination. M-MuLV reverse transcriptase (MBI Fermentas) was used for cDNA synthesis. The RT-PCR protocol is described above. Dynazyme DNA polymerase (Finnzymes, Espoo, Finland) was used to amplify cDNA template. Primer (20 pM/reaction) sequences for LacZ gene were SEQ ID NO. 1 for adenovirus and SE ID NO. 2 for baculovirus as forward primers, and SEQ ID NO. 3 for both viruses as a reverse primer. 39 cycles with 1 min denaturation (95° C.), 2 min annealing (57.5° C.) and 3 min extension (72° C.) times were used after a hot start (95° C. 5 min, 57.5° C. 3 min), followed by 10 min final extension at 72° C. 5 μl of the first PCR product was used for the second PCRwith forward primers SEQ ID NO.4 foradenovirus and SEQ ID NO. 5 for baculovirus. The reverse primer for both viruses was SEQ ID NO. 6. The same protocol was used as in the Example of WO-A-001/09390, but with 19 cycles. Bands were visualized on 1% agarose gel using ethidium bromide staining.

Clinical Chemistry Analyses

Clinical chemistry analysis from serum samples were done in Kuopio University Hospital Central Laboratory using routine clinical chemistry assays with Delta Pro V 5 equipment (Kone lnstruments Corporation).

In Vivo Tropism In Rat Brain

To analyze the gene transfer efficiency of baculovirus and adenovirus vectors, a total of 3×10⁸ pfu viruses was injected into corpus callosum of adult rats with the stereotaxic apparatus. The expression of transgene was analyzed 5, 10, 14 and 21 days after the gene transfer with X-gal staining and RT-PCR. Representative images of the transgene expression in the forebrain showed that both viruses lead to transgene expression in endothelial cells of brain microvessels throughout the forebrain. CD31 staining of serial sections showed positive cells in the same areas and with similar morphology, suggesting the transfection of endothelial cells. Baculoviruses showed a strong preference for choroid plexus cuboidal epithelial cells, whereas adenoviruses did not transduce these cells. In the first part of the experiments, the right corpus callosum just above the frontal horn of the lateral ventricle was chosen for the stereotaxic target point. To analyze how the injection site affects transduced cell types, the baculoviruses were also injected deeper into the parenchyma as described above. As a result, lacZ marker gene was mostly found in endothelial cells of the microvessels and in distinct choroid plexus cells in the third ventricle (2 mm from the injection site). Some transgene expression was also seen in the subarachnoidal space. The transgene expression was not detected in other cell types in brain. Clear differences were seen with the adenovirus vector; adenoviruses transduced ventricular ependymal lining and glial cells in corpus callosum with high efficacy. Cells in the subarachnoidal space were also occasionally transduced.

Gene Expression

Transduction efficiency of baculoviruses was as high as 76.8%±14 in choroid plexus epithelial cells. For adenoviruses the transduction efficiencies in corpus callosum and ependymal cells were 71.4%±9 and 83.5±11, respectively. Transgene expression was highest in the fifth day after the baculovirus transduction. The transgene expression decreased rapidly in two weeks: after 10 days 30.1%±6 of the choroid plexus cells were lacZ-positive and after 14 days, only a few positive cells could be detected. A similar time course was seen with adenoviruses: 5 days after the gene delivery, corpus callosum glial cells and ependymal cells were strongly lacZ-positive. The transgene expression decreased in 2-3 weeks, but remained detectable at three weeks timepoint (30.6%±13 for corpus callosum and 38.1%±12 for ependyma).

According to CD11b (OX-42) antibody-staining, baculoviruses did not induce a marked microglia response, since only 2 of 16/acZ-positive rats showed positive immunostaining in the brain. By contrast, adenovirus delivery elicited a marked microglia response within the transduced tissue. Microglia response was seen in all but one of the analyzed rats. The response increased from moderate to strong from day 5 to day 14 and stayed strong for 21 days.

Biodistribution and Clinical Chemistry

Representative nested RT-PCR analyses of the biodistribution samples showed that transgene expression was found in the fore and hindbrain after the local delivery and in the spleen, heart and lung from rats tested 5 days after the baculovirus gene transfer. After adenovirus injections, transgene expression was seen in forebrain and hindbrain and, from one animal of all tested, in the liver. No major safety problems were found in clinical chemistry analyses, which showed no significant effect of baculoviruses or adenoviruses on acetylaminotranserase, alanineaminotransferase, C-reactive protein, Creatinin, biliribin or Hgb values.

1. A method of treating a condition that can be mediated via the spinal cord or central nervous system (CNS) of a patient, comprising administering to the patient an effective amount of a baculovirus vector containing a gene, wherein the condition is treated by the action of said gene or a product thereof.

2. The method according to claim 1, wherein the condition requires enzyme replacement.

3. The method according to claim 1, wherein said gene is for endothelial NO synthase.

4. The method according to claim 3, wherein the condition is subarachnoid hemorrhage.

5. The method according to claim 2, wherein said gene is for endothelial NO synthase.

6. The method according to claim 5, wherein the condition is subarachnoid hemorrhage.


SEQUENCE LISTING (7 sequences)

SEQ ID NO: 1 - DNA, 20 bp, Artificial Sequence
  Description of Artificial Sequence Oligonucleotide
ttggaggcctaggcttttgc

SEQ ID NO: 2 - DNA, 20 bp, Artificial Sequence
  Description of Artificial Sequence Oligonucleotide
ttggcctagagtcgacggat

SEQ ID NO: 3 - DNA, 20 bp, Artificial Sequence
  Description of Artificial Sequence Oligonucleotide
tgaggggacgacgacagtat

SEQ ID NO: 4 - DNA, 22 bp, Artificial Sequence
  Description of Artificial Sequence Oligonucleotide
ggtagaagaccccaaggacttt

SEQ ID NO: 5 - DNA, 20 bp, Artificial Sequence
  Description of Artificial Sequence Oligonucleotide
ccaagaagaaacgcaaagtg

SEQ ID NO: 6 - DNA, 17 bp, Artificial Sequence
  Description of Artificial Sequence Oligonucleotide
cgccattcgccattcag

SEQ ID NO: 7 - DNA, 4495 bp, Artificial Sequence
  Description of Artificial Sequence Recombinant baculovirus
ccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagcttggcctagagtcgacggatccggggaattccccagtctcaggatccaccatggggcccaagaagaaacgcaaagtggggagcatgggggatcccgtcgttttacaacgtcgtgactgggaaaaccctggcgttacccaacttaatcgccttgcagcacatccccctttcccagctggcgtaatagcgaagaggcccgcaccgatcgcccttcccaacagttgcgcagcctgaatggcgaatggcgctttgcctggtttcgcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcagggggatcgcagatcggccagataccgatgctgccgcagcaaaaagcaggagcagatgccgccgtcgcagsaaagatgtcgcasaggaggaggcgatgctgccggcggaggaggcgaagtaagtagagggctgggctgggctgtgggggggtgtggggtgcgggactgggcagtctgggagtccctctcaccacttttcttacctttctaggatgctgccgtcgccgccgctcatacaccataaggttaaaaaaatactagatgcacagaatagcaagtccatcaaaactcctgcgtgagaattttaccagacttcaagagcatctcgccacatcttgaaaaatgccaccgtccgatgaaaaacaggagcctgctaagaaacaatgccacctgtcaataaatgttgaaaactcatcccattcctgcctcttggtccttgggcttggggaggggtgcgcggatgtggttagggaacatgactggtcaaatgggaagggcttcaaaagaattcccaatattgactaccaagccacctgtacagatctgcctgcatgctttgcatacttctgcctgctggggagcctggggactttccacaccctaactgacacacattccacagccggatctgaggaacccctagtgatggagttgg